(19) United States
(12) Patent Application Publication
(10) Pub. No.: US 2011/0152718 A1
(43) Pub. Date: Jun. 23, 2011

(54) DEVICE AND METHOD FOR EVALUATING THE HYDRIC LOSS OF A PERSON OR AN ANIMAL BY SWEATING

(75) Inventor: Frédéric REVOL-CAVALIER, Seyssins (FR)

(73) Assignee: COMMISSARIAT A L'ENERGIE ATOMIQUE ET AUX ENERGIES ALTERNATIVES, Paris (FR)

(21) Appl. No.: 12/974,432

(22) Filed: Dec. 21, 2010

(30) Foreign Application Priority Data
Dec. 21, 2009 (FR) 09 06204

(51) Int. Cl. A61B 5/00

(52) U.S. Cl. 600/573

(57) ABSTRACT

A device for evaluating the hydric loss of a person or an animal by sweating is provided with:
a support comprising a measuring face and a contact face designed to be applied on the skin of the person or animal,
a swelling element by absorption of sweat, securedly affixed to the support, said swelling element being arranged to be in direct contact or in fluidic communication with the skin and,
a measuring instrument to measure the increase of at least one dimension of the swelling element. Increase of the dimension of the swelling element is caused by absorption of said sweat.

A method for evaluating the hydric loss of a person or an animal using such a device is also described.

### BACKGROUND OF THE INVENTION

The invention relates to a device and a method for evaluating the hydric loss of a person or an animal by sweating.

### STATE OF THE ART

Numerous works have shown the interest of a good hydration for people in particular during physical sporting activities or for fragile people such as infants or elderly people. The hydric loss due to perspiration or lack of hydration can result in the appearance of physiological disorders, for example weight loss, concentration problems, excessive fatigue or dizziness. For the most severe cases of dehydration, losses of intellectual faculties or physiological disorders able to cause the death of the person or of the animal can also occur.

Sweating is the main motor in thermal regulation of the human body and that of certain higher-order animals. When sweating becomes insufficient to cool the body, the internal body temperature increases. To prevent dehydration or over-hydration, one approach consists in monitoring the internal body temperature using conventional body temperature measurement techniques. External body temperature measurements, for example axillary measurement, are not sufficiently precise and quick for such monitoring. Furthermore, internal body temperature measurements, for example oral, rectal or auricular, have a limited use, limited to the laboratory and to the medical field, and are not suitable for continuous use in real time.

Another approach consists in evaluating the hydric loss by sweating i.e. the quantity of sweat perspired over a given time. This evaluation is conventionally made by weighing on a differential balance. The person is weighed several times throughout a physical exercise or during a check-up to be able to calculate the ratio between the hydric loss and the weight of the person. This ratio is characteristic of the degree of dehydration of the person and consequently of his/her state of hydration. Nevertheless, this solution is difficult to implement outside a laboratory or outside a medical environment. This solution is precise and sensitive, but only remains applicable in a laboratory and cannot be suitable for mobile applications.

Recent works have proposed devices enabling the hydric loss of a person to be evaluated from measurement of the moisture content near a person's skin. For example, the documents GBA-2452258, WO2005120333, U.S. Pat. No. 4,965,698, JP-A-2002263072 and U.S. Pat. No. 3,350,941 describe devices applied to the surface of a person's skin, arranging a measuring space above the skin and containing a moisture sensor to measure the moisture contained in the atmosphere, said measurement being representative of the secreted sweat.

### OBJECT OF THE INVENTION

The object of the invention is to provide a precise and reliable device carried by a person or an animal, whereby the hydric loss by sweating of the person or animal can be evaluated continuously in real time.

According to the invention, this object is achieved by a device for evaluating the hydric loss of a person or an animal by sweating and a method using one such device according to the appended claims.

In particular, this object is achieved by a device provided with:
a support comprising a measuring face and a contact face designed to be applied to the skin of the person or animal,
a swelling element, securedly affixed to the support, that swells by absorption of sweat, said swelling element being arranged to be in direct contact or in fluidic communication with the skin and,
a measuring instrument for measuring the increase of at least one dimension of the swelling element, said increase being caused by absorption of said sweat.

### BRIEF DESCRIPTION OF THE DRAWINGS

Other advantages and features will become more clearly apparent from the following description of particular embodiments of the invention given for non-restrictive example purposes only and represented in the appended drawings, in which:

FIG. 1 schematically represents, in cross-section, a particular embodiment of a device for evaluating the hydric loss of a person or an animal by sweating according to the invention.

FIG. 2 schematically represents, in cross-section, a step of an embodiment of a method using the device according to FIG. 1.

FIGS. 3 to 5 schematically represent, in top view, particular embodiments of a device according to FIGS. 1 and 2.

FIG. 6 schematically represents, in cross-section, a second particular embodiment of a device according to the invention.

FIG. 7 schematically represents, in cross-section, a step of an embodiment of a method using a device according to FIG. 6.

FIG. 8 schematically represents, in cross-section, an alternative embodiment of the device according to FIG. 7.

FIG. 9 schematically represents, in cross-section, a third particular embodiment of a device according to the invention.

FIG. 10 schematically represents, in cross-section, a step of an embodiment of a method using a device according to FIG. 9.

FIG. 11 schematically represents, in cross-section, an alternative embodiment of the device according to FIG. 10.

FIG. 12 schematically represents, in cross-section, an operating assembly for calibrating a device according to the invention.

FIG. 13 represents the electronic diagram of the assembly according to FIG. 12.

FIG. 14 is a graph representing the voltage measured from the assembly of FIG. 12 and according to the electronic diagram of FIG. 13 versus the volume of water absorbed.

### DESCRIPTION OF PARTICULAR EMBODIMENTS

The object of the invention is to provide a device designed for an embedded use by any person and animal having sweat glands and able to perspire. The device enables the hydric loss of the person or animal by sweating to be evaluated from an estimation of the quantity of sweat perspired by the person or the animal. The device is preferably used continuously and in real time for direct reading of the hydric loss evaluation.

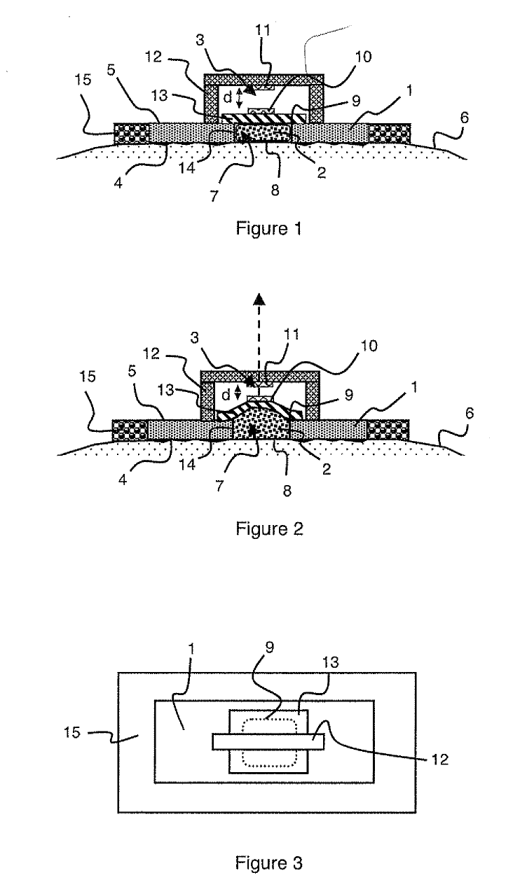

According to a first particular embodiment represented in FIGS. 1 to 5, a device is provided with a support 1, with a swelling element 2 and with a measuring instrument 3 for measuring the increase of at least one dimension of the swelling element 2.

Support 1 comprises a contact face 4 and a measuring face 5. Contact face 4 is designed to be applied on the skin 6 of the person or of the animal.

Measurement of the increase of at least one dimension of swelling element 2 is performed on the side where measuring face 5 is located.

Support 1 is preferably formed by any known material sufficiently supple to be able to adapt to the outline of the body on which it is applied and having sufficient strength to support the elements constituting the device, Support 1 is further inert to sweat and preferably dermatologically acceptable.

Swelling element 2 is securedly affixed to support 1 and is able to swell by absorption of sweat. Swelling element 2 is arranged to be in direct contact or in fluidic communication with the localized area of skin 6 covered by the device, so that the sweat given off by sweating at the level of this area is absorbed by swelling element 2. What is meant by fluidic communication is the fact that the sweat perspired by skin 6 is conveyed by any known method from skin 6 to swelling element 2. Swelling element 2 absorbs the sweat given off in liquid form. Sweat on vapor form, in proximity to the surface of the localized area to be absorbed, can also be absorbed by swelling element 2. So, sweat on vapor form and on liquid form can both be absorbed by the swelling element 2.

Swelling element 2 advantageously comprises a hydrophilic polymer. The hydrophilic polymer is preferably chosen from synthetic hydrophilic polymers, natural polysaccharides, semi-synthetic polysaccharides and derivatives of cellulose and their salts.

For example purposes, the natural polysaccharides can be alginates, xanthane rubber, guar, gum arabic or carob.

The semi-synthetic polysaccharides are preferably chosen from cellulose derivatives, for example from methylhydroxyethylcellulose, carboxymethylcellulose and its salts such as sodic carboxymethylcellulose or calcic carboxymethylcellulose, hydroxypropylcellulose, hydroxypropylmethylcellulose and mixtures of hydroxypropylcellulose and hydroxypropylmethylcellulose.

The hydrophilic polymer can also be chosen from polyvinylpyrrolidones, amino acid polymers such as polylysines, and advantageously from polymers derived from acrylic and methylacrylic acids and their salts. The hydrophilic polymer is preferably a polyacrylate or a polyacrylate salt.

Swelling element 2 can be formed by a single hydrophilic polymer cited in the above or by a mixture of several of them.

In order to foster rapid increase of the volume of swelling element 2, with the hydrophilic polymers above-mentioned, swelling element 2 can comprise swelling stimulation agents. The swelling stimulation agents enhance hydration of the polymer structures of swelling element 2 and/or reduce the interactions between the polymer structures of swelling element 2 and the sweat constituents. Hydrophilic diluents such as lactose, mannitol, sorbitol and microcrystalline cellulose can also be used for this purpose. Substances favouring wettability of the polymer structure of swelling element 2 can also be used such as sodium laurylsulphate, sodium rinoleate, sodium teradecylsulphate, sodium dioctylsulphosulphonate, cetomagrocol, poloxamer, polysorbates or any other dermatologically acceptable tensioactive.

Swelling element 2 can also advantageously comprise textile fibers such as cotton, to uniformly distribute sweat within swelling element 2 and to ensure uniform swelling of the hydrophilic polymer.

The measuring instrument 3 is preferably a sensor or a detector disposes at the level of measuring face 5 to determine the increase of at least one dimension of swelling element 2 caused by absorption of sweat. Swelling element 2 and measuring instrument 3 are two distinct elements in the device with different functions. Swelling element 2 absorbs the sweat perspired by skin 6, for the most part in liquid form, and measuring instrument 3 determines the increase of volume of swelling element 2 caused by absorption of the perspired sweat.

Measuring instrument 3 is advantageously chosen from photodetectors, capacitive sensors, inductive sensors, mechanical position sensors and sensors of strain gage type.

Measuring instrument 3 is conventionally connected to a data processing and display system (not shown). The data are for example stored and/or transmitted by hardwired or non-wired means to an electronic circuit, and are then processed and displayed by any known method.

As represented in FIGS. 1 and 2, support 1 comprises a through hole 7 opening out onto contact face 4 and onto measuring face 5 respectively via a first opening 8 and a second opening 9. Swelling element 2 is housed and stressed in through hole 7 so that an increase of a dimension of swelling element 2 makes element 2 protrude out from through hole 7. Swelling element 2 is salient from through hole 7 mainly on the side where measuring face 5 is located, as protrusion of swelling element 2 on the side where contact face 4 is located is hampered by the presence of skin 6, contact face 4 being applied on the localized area of skin 6 (FIG. 2).

Measuring instrument 3 is advantageously arranged at the level of measuring face 5 to measure the increase of the dimension of swelling element 2 salient from through hole 7 on the side where measuring face 5 is located. Measuring instrument 3 thereby enables the protrusion of swelling element 2 from through hole 7 on the measuring face 5 side to be measured, this protrusion being proportional to the quantity of sweat absorbed.

Measuring instrument 3 is for example formed by a capacitive sensor. In particular, measuring instrument 3 comprises a capacitor formed by a mobile electrode 10 and a fixed electrode 11 facing said mobile electrode 10. The mobile and fixed electrodes, respectively 10 and 11, are preferably made from metal. A free space separates mobile electrode 10 and fixed electrode 11 by a distance d. Mobile electrode 10 is arranged above swelling element 2 and is securedly attached to said element 2 so that an increase of at least one dimension of swelling element 2 causes movement of mobile electrode 10 towards fixed electrode 11.

As represented in FIG. 1, the device can comprise an armature 12 situated on the measuring face 5. Measuring instrument 3 can in particular be arranged above the swelling element 2. Measuring instrument 3 can be supported by armature 12. In particular, at least a part of measuring instrument 3 is supported by armature 12. In this way, fixed electrode 11 can be supported by armature 12 which positions fixed electrode 11 above mobile electrode 10, itself situated above second opening 9 (at the top in FIG. 1).

Measuring face 5 comprises a confinement element 13 of swelling element 2 salient from through hole 7. Confinement element 13 can be formed by a deformable membrane fixed to measuring face 5 and covering at least a part of swelling element 2. Confinement element 13 is advantageously secured at the level of at least two overlap points of confinement element 13 with measuring face 5.

As represented in FIG. 2, when the device is applied to a localized area of skin 6, first opening 8 is facing skin 6 and blocked off by the latter. Swelling element 2 is contained by side walls 14 of through hole 7. Absorption of the sweat collected at the level of the area of skin 6 covered by swelling element 2 causes expansion of swelling element 2 out of through hole 7. Swelling element 2 is then salient from the surface of measuring face 5 via second opening 9.

As represented in FIG. 2, mobile electrode 10 is securedly affixed to swelling element 2 for expansion of swelling element 2 by absorption of sweat to induce movement of mobile electrode 10 towards fixed electrode 11.

Expansion of swelling element 2 makes mobile electrode 10 move towards fixed electrode 11 and reduces the distance d between the electrodes, respectively 10 and 11. Variation of distance d leads to a variation of the capacitance of the capacitor formed by the two fixed and mobile electrodes, respectively 10 and 11. The increase of a dimension, for example the height, of swelling element 2 in FIG. 2, is thus transcribed into a physically measurable quantity: the capacitor capacitance. The capacitance is then proportional to the volume of sweat absorbed. Monitoring of the capacitance value of the capacitor enables the volume of sweat absorbed versus time to be measured and consequently enables the hydric loss of the person or animal to be deduced therefrom.

Mobile electrode 10 can advantageously be deposited on the membrane constituting confinement element 13. Confinement element 13 is arranged between mobile electrode 10 and swelling element 2. Fixed electrode 11 is situated on the expansion path of swelling element 2 (dashed arrow in FIG. 2).

Confinement element 13 accompanies expansion of swelling element 2 out of through hole 7, while at the same time containing it to circumscribe its path and to prevent it from being dispersed. Confinement element 13 thereby enables swelling element 2 to be contained and the salient part of swelling element 2, i.e. the part situated outside through hole 7, to be confined.

Support 1 advantageously comprises means for fixing 15 the device onto skin 6 or onto the body of the person or animal. Support 1 can for example comprise one or more adhesives.

As represented in FIG. 3, the device can be secured by an adhesive situated at the periphery of the device. The adhesive maintains the device on the localized area of skin 6 where measurement is to be made.

According to an alternative embodiment, not shown, means for fixing 15 can be formed by fasteners designed to embrace a part of the body of the person or animal. The fasteners can be any known mechanical securing means capable of securing the device around a part of the body of the person or animal. Strips of Velcro® type, straps, arm-bands or dorsal support belts can be cited for example purposes.

According to another alternative embodiment, not shown, a part or all of contact face 4 can be adhesive.

As represented in FIG. 3, confinement element 13 is preferably arranged above swelling element 2 to cover second opening 9 and a part of support 1 at the periphery of second opening 9. Armature 12 can be in the shape of an arch bearing on each side of second opening 9.

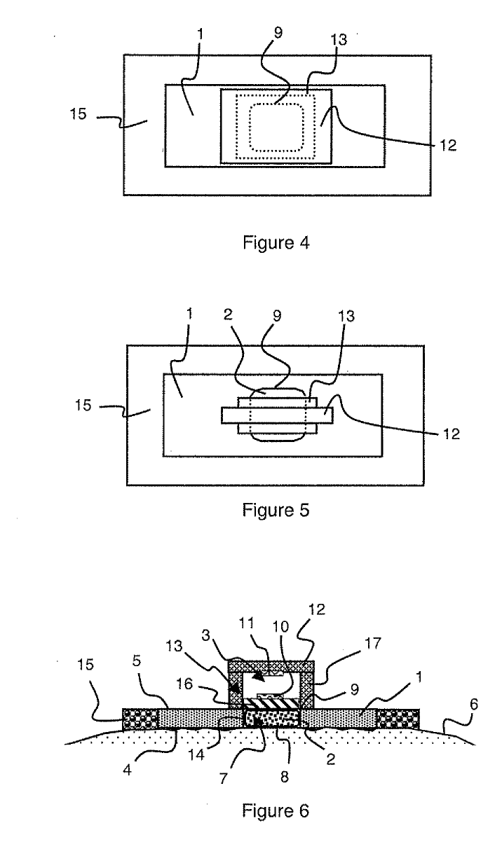

According to an alternative embodiment represented in FIG. 4, armature 12 can be formed by a cover bearing on the whole of the periphery of second opening 9 and then creates a closed space enclosing measuring instrument 3.

According to another alternative embodiment represented in FIG. 5, confinement element 13 only covers a part of second opening 9, leaving a part of swelling element 2 uncovered. The membrane forming confinement element 13 is fixed only on each side of second opening 9 (to the right and the left in FIG. 5).

According to a particular embodiment, a method for evaluating the hydric loss of a person or animal by sweating comprises application of the evaluation device described above on a localized area of skin 6 of the person or animal, contact face 4 being positioned facing skin 6 and swelling element 2 being in contact or in fluidic communication with skin 6 (FIG. 1). While maintaining the device in place, the increase of at least one dimension of swelling element 2 with time is then measured by means of the measuring instrument 3 by any known method (FIG. 2).

The increase of a single dimension of swelling element 2 is advantageously measured with time. This dimension can be the height, depth, length or width.

Nevertheless, measurement can also be made on two dimensions, for example on a surface, or even on three dimensions, for example on a volume.

Measurement of the increase of at least one dimension of swelling element 2 with time enables the quantity of sweat emitted at the level of the localized area of skin 6 of the person or animal to be determined versus time.

The results obtained are advantageously extrapolated to give a global hydric loss. Extrapolation can take account in particular of the surface ratio between the localized area of skin 6 covered by the device and the total surface of skin 6 of the body. It can also take account of the position of the sensor on the human body, certain areas of the body, in particular the thigh, being preferred for positioning the sensor. Extrapolation can also take account of the results of a calibration, calibration being able to be performed from a reference method such as weighing. Monitoring the increase of at least one dimension enables the hydric loss of the person or animal to be evaluated in real time and continuously.

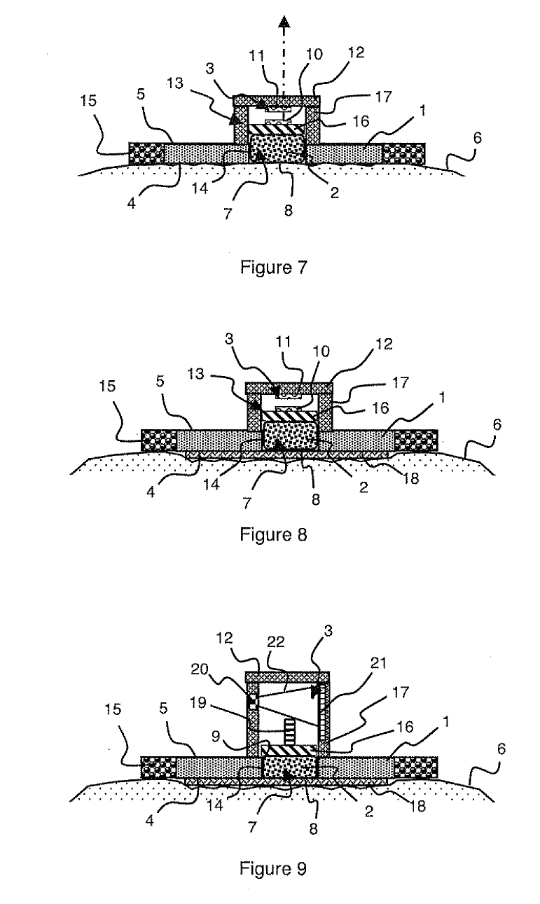

According to a second embodiment represented in FIGS. 6 and 7, the device is identical to that of the first particular embodiment described above with the exception of the confinement element 13. According to this particular embodiment, confinement element 13 is formed by a rigid or semi-rigid layer 16 which operates in conjunction with guiding elements 17. Rigid or semi-rigid layer 16 is arranged above swelling element 2 and between guiding elements 17. Guiding elements 17 are salient from the surface of measuring face 5. Rigid or semi-rigid layer 16 is mobile along said elements 17.

Guiding elements 17 can consist of rims situated on the same side as measuring face 5, around second opening 9, and delineating rigid or semi-rigid layer 16.

Armature 12 can comprise guiding elements 17 thereby forming a single assembly. Alternatively, armature 12 can consist of a cover placed on guiding elements 17 (at the top in FIG. 6).

As represented in FIG. 7, due to the effect of expansion of swelling element 2, rigid or semi-rigid layer 16 moves in translation along an expansion path of swelling element 2 represented by the dashed arrow.

According to an alternative embodiment, not shown, guiding elements 17 can comprise grooves of slide type in which rigid or semi-rigid layer 16 slides.

According to an alternative embodiment represented in FIG. 8, contact face 4 comprises a drainage layer 18 of the sweat to swelling element 2. Once the device has been applied on the localized area of skin 6, drainage layer 18 is in contact with skin 6 and in fluidic communication with swelling element 2. The sweat perspired at the level of this localized area is drained to swelling element 2, for example by capillarity or by means of a fluidic network formed by any known method. Swelling element 2 is not in direct contact with skin 6.

Drainage layer 18 can be composed of a hydrophilic part which will drain the sweat to swelling element 2. Drainage layer 18 can also comprise a hydrophobic part, for example made from cellulose or polyester, to prevent absorption of sweat at the level of certain areas of contact face 4.

According to an alternative embodiment, not shown, drainage layer 18 can be formed by a layer having a larger surface area than that of contact face 4 so that drainage layer 18 extends on skin 6 of the person or animal beyond support 1.

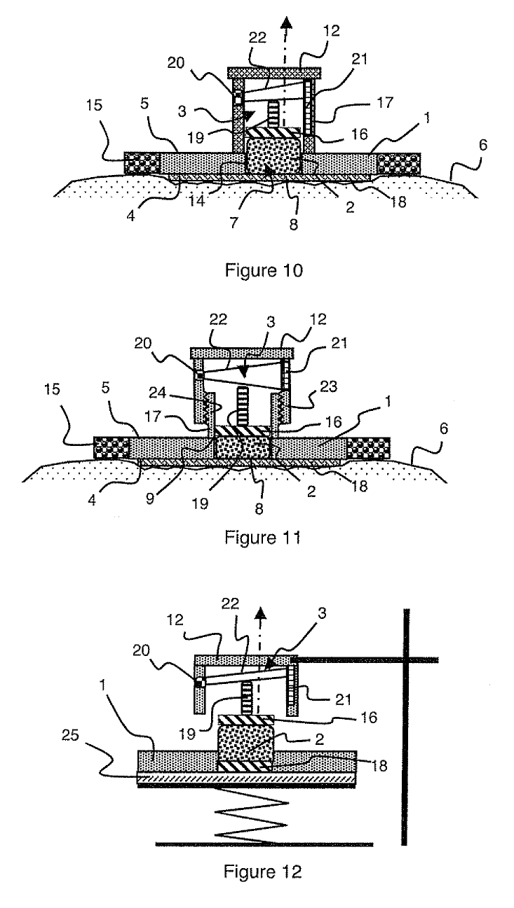

According to a third embodiment represented in FIGS. 9 and 10, measuring instrument 3 comprises an optic cache 19 and a photodetector formed by a photodiode 20 and a detection window 21. According to this particular embodiment, armature 12 comprises guiding elements 17 and is designed both to support measuring instrument 3 and to guide rigid or semi-rigid layer 16. Photodiode 20 and detection window 21 are situated on the inner side wall of armature 12. Detection window 21 is facing photodiode 20, on the optic path of beam 22 emitted by photodiode 20.

As represented in FIG. 9, optic cache 19 is securedly affixed to swelling element 2. Optic cache 19 can be fixed to the free top surface of rigid or semi-rigid layer 16 (at the top in FIG. 9). Optic cache 19 has a prominent part protruding out from measuring face 5. The prominent part is designed to cross the optic path of beam 22 emitted by photodiode 20.

The electric signal from the photodetector varies proportionally to the quantity of light received from optic beam 22. The increase of the dimension of swelling element 2 induces movement of optic cache 19 in vertical translation (from bottom to top in FIG. 10). This movement brings optic cache 19 between photodiode 20 and detection window 21 to progressively blank off optic beam 22.

As represented in FIG. 10, when expansion of swelling element 2 takes place, the prominent part of optic cache 19 is positioned between photodiode 20 and detection window 21 to blank off at least a part of optic beam 22. The intensity of the electric signal from the photodetector is inversely proportional to the increase of the dimension of swelling element 2, in the heightwise direction (from bottom to top in FIGS. 9 and 10) and is by consequently proportional to the quantity of sweat absorbed by swelling element 2.

According to an alternative embodiment represented in FIG. 11, armature 12 comprises a bottom open part 23 (at the bottom in FIG. 11) which fits onto top part 24 of guiding elements 17 (at the top in FIG. 11). This fitting secures armature 12 to support 1. This fitting also enables optic cache 19 to be positioned with respect to the photodetector. This fitting in particular enables the photodetector to be reset before the evaluation device is applied to skin 6. Resetting corresponds to setting to a known and reproducible capacitance or voltage value delivered by the photodetector, before measuring instrument 3 is used. Setting is performed by any known method.

For illustration purposes represented in FIG. 11, the inner wall of bottom open part 23 of armature 12 and the outer wall of top part 24 of guiding elements 17 are threaded to form a screw thread and to enable bottom open part 23 of armature 12 to be screwed onto top part 24 of guiding elements 17. Tightening or loosening of bottom open part 23 positions optic cache 19 vertically. The value of the electric voltage collected at the output of the measuring instrument 3 is then adjusted according to the optic path taken by photodiode 20, by tightening or loosening bottom open part 23 of armature 12, to achieve an initial reference value.

This variant can be applied to the measuring instrument 3 comprising a capacitor as described in the second embodiment. Adjustment then enables fixed electrode 11 to be positioned with respect to mobile electrode 10 to obtain an initial reference value.

According to a fourth particular embodiment, not shown, the deformation of swelling element 2 is measured by means of a sensor of strain gage type. What is meant by strain gage sensor is a sensor measuring a mechanical deformation using electrical means. In this case, the strain gage can be applied directly on at least one free surface of swelling element 2. The strain gage is for example in the form of a coil made from conductive material, for example a metallic material or a conductive polymeric material, the electrical resistance whereof is measured, a resistance which varies as the gage is deformed. The strain gage can be formed directly on swelling element 2 or be placed on swelling element 2, for example by sticking, by means of any known method. The embodiment is particularly advantageous as it enables the sensor assembly to be fitted directly on swelling element 2 and does not require precise adjustment between a fixed part and a mobile part. Such an embodiment further presents a simpler design than the embodiments described in the foregoing and can be achieved at lower cost.

The evaluation method can also implement usual and commonplace calibration practices. What is meant by calibration is prior establishment of one or more calibration curves under similar conditions, for one or more temperatures comprised between 35° C. and 40° C. and in particular around 37° C. and for known quantities of sweat or of a liquid such as water having physicochemical properties close or similar to those of sweat. The value(s) obtained from the embedded device borne by the person or animal are compared with the calibration curves.

### Example of Calibration

### Fabrication of a Calibration Device

A device, represented in FIG. 12, comprises a stack of two cellulose films with a surface of 1 cm² respectively forming drainage layer 18 and semi-rigid layer 16. A mixture of 20 mg of polyacrylate and cotton forms swelling element 2 and is arranged between the two cellulose films 16 and 18. The stack formed is fixed onto a glass plate 25 by the support 1 consisting of an adhesive. An optic cache 19 consisting of aluminum adhesive tape is stuck onto the free surface of the cellulose film forming semi-rigid layer 16.

As represented in FIG. 12, a photodetector marketed by OMROM under the reference EE-SG3, is placed above optic cache 19 so that the optic cache 19 is located on the optic path of beam 22 emitted by photodetector.

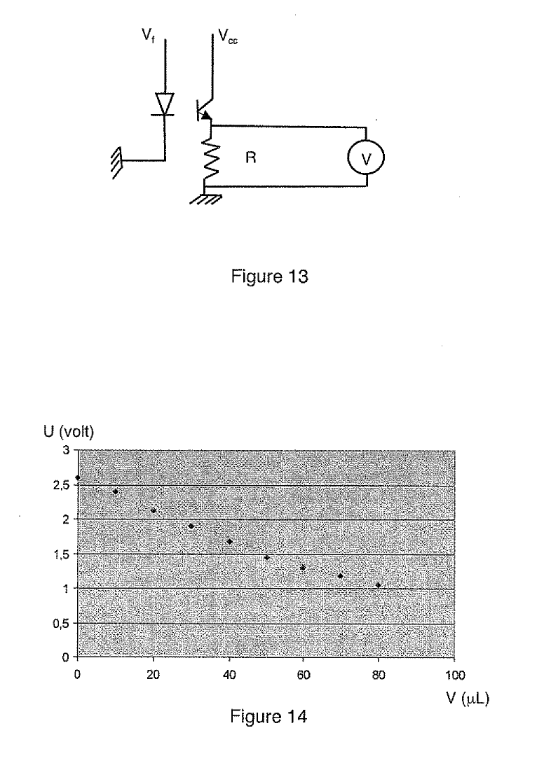

An electronic assembly, represented in FIG. 13, is made to monitor the variation of the voltage at the terminals of a 1 KΩ resistance, noted R, with the following imposed parameters:

V_{f}=1.1 V and

V_{cc}=3V.

A predefined quantity of water, noted V, is deposited on swelling element 2. The variation of the voltage at the terminals of resistance R is then measured. The results are set out in the table below:

| Water volume absorbed in μL | Measured voltage in Volts |
|---|---|
| 0 | 2.6 |
| 10 | 2.4 |
| 20 | 2.13 |
| 30 | 1.9 |
| 40 | 1.67 |
| 50 | 1.45 |
| 60 | 1.3 |
| 70 | 1.18 |
| 80 | 1.05 |

The calibration curve plot U=f(V) is represented in FIG. 14. It can be observed that the variation of voltage U measured at the terminals of resistance R is proportional to the quantity of water V absorbed by swelling element 2. Calibration curve plot U=f(V) then enables the quantity of sweat absorbed over a given time to be determined from a voltage measurement made from an evaluation embedded device borne by a person or an animal and similar to the calibration device (same swelling element).

The evaluation device and method according to the invention enable a continuous evaluation of the hydric loss of a person or animal to be made in real time. The device according to the invention can be embedded i.e. borne by a person or an animal. Use of device according to the invention is not limited to use in a laboratory and can be used by a person unfamiliar with the hospital environment. The evaluation method is moreover easy to implement and gives reliable and sensitive results enabling rapid intervention on the causes of dehydration of the person or animal.

1. A device for evaluating the hydric loss of a person or an animal by sweating, provided with:
a support comprising a measuring face and a contact face designed to be applied to the skin of the person or animal,
a swelling element, secure affixed to the support, that swells by absorption of sweat, said swelling element being arranged to be in direct contact or in fluidic communication with the skin and,
a measuring instrument for measuring the increase of at least one dimension of the swelling element, said increase being caused by absorption of said sweat.

2. The device according to claim 1, wherein the swelling element comprises a hydrophilic polymer.

3. The device according to claim 2, wherein the hydrophilic polymer is chosen from synthetic hydrophilic polymers, natural polysaccharides, semi-synthetic polysaccharides and derivatives of cellulose and their salts.

4. The device according to claim 2, wherein the hydrophilic polymer is chosen from polyvinylpyrrolidones, polymers derived from acrylic and methylacrylic acids and their salts, and amino acid polymers.

5. The device according to claim 1, wherein the swelling element comprises swelling stimulation agents of said element.

6. The device according to claim 1, wherein the measuring instrument is chosen from photodetectors, capacitive sensors, inductive sensors, mechanical position sensors and sensors of strain gage type.

7. The device according to claim 1, comprising an armature situated on the measuring face and wherein at least a part of the measuring instrument is supported by said armature.

8. The device according to claim 1, wherein the contact face comprises a drainage layer of the sweat to the swelling element.

9. The device according to claim 1, wherein the support comprises a through hole opening out onto the contact face and onto the measuring face in which the swelling element is housed and wherein the measuring instrument is arranged at the level of the measuring face.

10. The device according to claim 9, wherein the swelling element is housed and stressed in the through hole so that an increase of a dimension of the swelling element makes said element protrude out from the through hole and wherein the measuring instrument is arranged above the through hole.

11. The device according to claim 10, wherein the measuring face comprises a confinement element of the swelling element salient from the through hole.

12. The device according to claim 11, wherein the confinement element is formed by a deformable membrane fixed to the measuring face and covering at least a part of the swelling element.

13. The device according to claim 11, wherein the confinement element is formed by a rigid or semi-rigid layer arranged above the swelling element which operates in conjunction with guiding elements salient from the surface of the measuring face and wherein said layer is mobile along the guiding elements.

14. A method for evaluating the hydric loss of a person or an animal by sweating, wherein an evaluation device according to claim 1 is applied on a localized area of the skin of the person or animal, the contact face being positioned facing the skin and the swelling element being in contact or in fluidic communication with the skin and wherein the increase of at least one dimension of the swelling element is then measured over time by means of the measuring instrument, while at the same time keeping said device in place.

15. The method according to claim 14, wherein the increase of a single dimension of the swelling element is measured over time.

16. The method according to claim 14, wherein the contact face comprises a drainage layer of the sweat and wherein, once the device has been applied on the localized area of the skin, the drainage layer is in contact with the skin and in fluidic communication with the swelling element.
